(19) United States
(12) Patent Application Publication
(10) Pub. No.: US 2008/0255599 A1
(43) Pub. Date: Oct. 16, 2008

(54) ACUPUNCTURE NEEDLE

(75) Inventors: Shigeaki IDE, Shizuouka-ken (JP); Tomoya Hasegawa, Tokyo (JP); Satoshi Fukasawa, Shizuouka-ken (JP); Hiroaki Nanba, Shizuouka-ken (JP)

Correspondence Address: EGBERT LAW OFFICES, 412 MAIN STREET, 7TH FLOOR, HOUSTON, TX, 77002, US

(73) Assignee: TOYO RESIN Corporation, Fuji-shi (JP)

(21) Appl. No.: 12/053,151

(22) Filed: Mar. 21, 2008

(30) Foreign Application Priority Data
Apr. 10, 2007 (JP) 2007-102459

(51) Int. Cl. A61B 17/34

(52) U.S. Cl. 606/189

(57) ABSTRACT

An acupuncture needle gives skin a favorable stimulus without invading the skin when in contact with or pressed against the skin. The acupuncture needle includes a base plate and projections formed on a surface of the base plate integrally therewith. Each of the projections have elasticity and includes a base portion and a substantially cylindrical head portion formed on the base portion. The head portion has a flat top surface substantially parallel to the surface of the base plate and a side perpendicular to the surface f the base plate. The base portion has an inwardly depressed external curved line in a central vertical section and a diameter in a transverse cross section gradually becoming larger toward the surface of the base plate. The projections sway in a direction substantially parallel to the skin when the acupuncture needle is in contact with or pressed against the skin.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

1. Field of the Invention

The present invention relates to an acupuncture needle or an acupuncture means. More particularly, the present invention relates to an acupuncture needle effective in easing or removing numbness of the sole of a foot, numbness of upper limbs or lower limbs, pain in interphalangeal joints, swelling, scapulohumeral periarthritis, headache, lumbago, arthralgia, myosalgia, stiffness in the shoulders, etc.

2. Description of Related Art Including Information Disclosed Under 37 CFR 1.97 and 37 CFR 1.98

The acupuncture needle is a needle means for stimulating the skin without invading or cutting into the skin when the acupuncture needle is in contact with or pressed against the skin.

Japanese Utility Model Publication No. 3120940 discloses an acupuncture needle comprising a base plate and many projections formed on a surface of said base plate.

The projections have a diameter of 0.01 to 0.20 mm and a length of 0.1 to 2.0 mm, said projections being disposed at intervals of 0.1 to 5.0 mm.

Japanese Utility Model Publication No. 3120940 discloses projections having various shapes. These projections have a thin or round end, or have an overall shape of a truncated cone. The projections having a thin or round end is likely to invade the skin. The projections having an overall shape of a truncated cone may be broken or bent.

### BRIEF SUMMARY OF THE INVENTION

It is an object of the invention to provide an acupuncture needle, which has obviated all of the above-mentioned disadvantages of the conventional acupuncture needle.

It is another object of the invention to provide an acupuncture needle, which gives the skin a favorable stimulus without invading the skin and without being broken or bent.

As a result of earnest study, the inventors have found that, to give the skin a favorable stimulus without invading the skin and without being broken or bent, projections of an acupuncture needle must “sway” when the acupuncture needle is in contact with or pressed against the skin.

The term “sway” in the present invention means to move in a direction substantially parallel to the skin.

These and other objects have been attained by the following acupuncture needle.

The acupuncture needle of the present invention includes a base plate and many projections formed on a surface of said base plate integrally therewith. Each of said projections have elasticity and comprise a base portion and a substantially cylindrical head portion formed on said base portion. The head portion has a flat top surface substantially parallel to said surface of said base plate and a side perpendicular to said surface of said base plate. The base portion has an inwardly depressed external curved line in a central vertical section and a diameter in a transverse cross section gradually becoming larger toward said surface of said base plate. Thus, each of said projections sway in a direction substantially parallel to the skin when the acupuncture needle is in contact with or pressed against the skin.

Said flat top surface of said head portion has an inclination of below 9° with respect to said surface of said base plate.

Said flat top surface of said head portion covers above 85% of the surface area of said head portion excluding said side.

Said base plate and said projections are made of a material having a surface hardness of 80 to 120 by Rockwell hardness test or durometer hardness test.

Said head portion has a diameter of 10 to 70 μm.

The distance between said surface of said base plate and said top surface of said head portion is 50 to 500 μm.

The length of said side of said head portion perpendicular to said surface of said base plate is 5 to 50μ and below 10% of said distance between said surface of said base plate and said top surface of said head portion.

According to the present invention, the projections of the acupuncture needle sway in a direction substantially parallel to the skin by slight movements of the skin when the acupuncture needle is in contact with or pressed against the skin. Therefore, the acupuncture needle gives the skin a favorable stimulus without invading the skin when the acupuncture needle is in contact with or pressed against the skin. As a result, the acupuncture needle eases or removes the numbness of the sole of a foot, the numbness of upper limbs or lower limbs, pain in interphalangeal joints, swelling, scapulohumeral periarthritis, headache, lumbago, arthralgia, myosalgia, stiffness in the shoulders, etc.

### BRIEF DESCRIPTION OF THE SEVERAL VIEWS OF THE DRAWINGS

FIG. 1 is a sectional view showing an example of the acupuncture needle according to the present invention.

FIG. 2 is a front view showing a portion of a projection.

FIG. 3 is a sectional view taken on line III-III in FIG. 2.

FIG. 4 is a sectional view taken on line IV-IV in FIG. 2.

FIG. 5 is a front view showing a modified example of a portion of a projection.

FIG. 6 is a front view showing an undesirable example of a projection.

FIG. 7 is a front view showing another undesirable example of a projection.

FIG. 8 is a front view showing a further undesirable example of a projection.

FIG. 9 is a front view showing a further undesirable example of a projection.

FIG. 10 is a schematic view showing another example of the acupuncture needle according to the present invention.

FIG. 11 is a perspective view showing said acupuncture needle attached to a supporting means.

FIG. 12 is an elevation view showing said acupuncture needle which is in contact with or pressed against the skin.

FIG. 13 is a perspective view showing said acupuncture needle attached to an adhesive sheet.

FIG. 14 is a sectional view showing said acupuncture needle which is in contact with or pressed against the skin.

FIG. 15 is a front view showing the sway of a projection.

### DETAILED DESCRIPTION OF THE INVENTION

The present invention will now be described in detail with reference to the attached drawings.

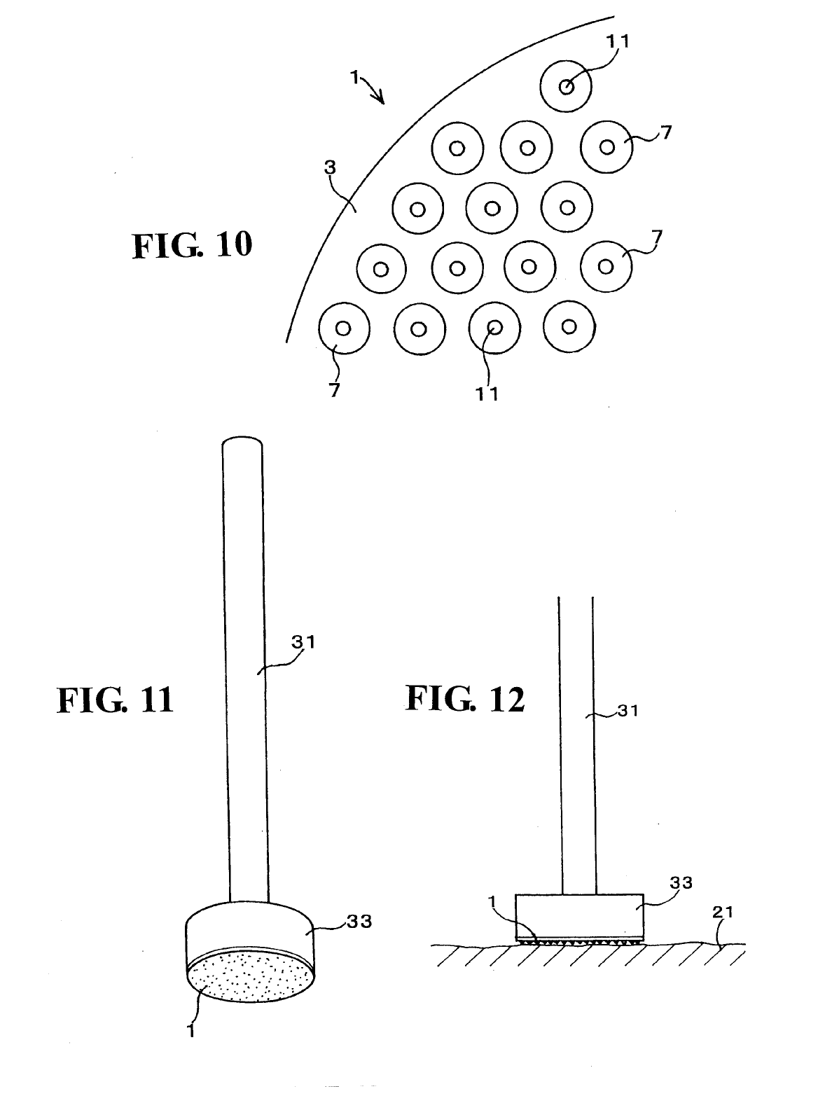

An acupuncture needle 1 according to the present invention comprises a base plate 3 and many projections 7 formed on a surface 5 of said base plate 3 integrally with said base plate 3. Said base plate 3 is preferably a circular plate. See FIG. 10.

Each of said projections 7 has elasticity.

Each of said projections 7 comprises a base portion 9 and a substantially cylindrical head portion 11 formed on said base portion 9.

Said head portion 11 has a flat top surface 13 substantially parallel to said surface 5 of said base plate 3. Said head portion 11 has a side 15 perpendicular to said surface 5 of said base plate 3.

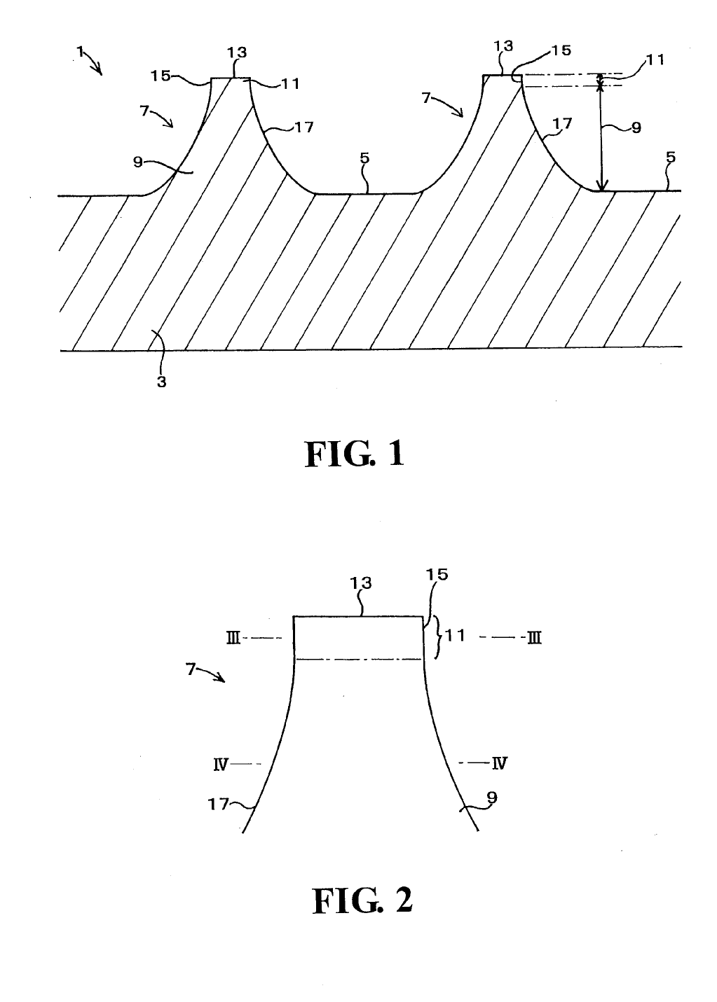

Said base portion 9 has an inwardly depressed external curved line 17 in a central vertical section (FIG. 1), said base portion 9 having a diameter in a transverse cross section (FIG. 4) gradually becoming larger toward said surface 5 of said base plate 3.

Thereby, each of said projections 7 sways in a direction substantially parallel to the skin 21 when the acupuncture needle 1 is in contact with or pressed against the skin 21. The sway of each projection 7 is caused by slight movements of the skin.

Each projection 7 does not move by itself. Each projection 7 is stationary when it is out of contact with the skin 21.

Since said projections 7 have elasticity, they spring back to their original shape when they return to a stationary condition after swaying.

When each of said projections 7 sways, the base portion 9 slightly moves and the head portion 11 does not change its shape. See FIG. 15.

Said flat top surface 13 of said head portion 11 has an inclination of below 9° with respect to said surface 5 of said base plate 3. In other words, the angular error in parallelism of said flat top surface 13 of said head portion 11 with respect to said surface 5 of said base plate 3 must be below 10%. If the flat top surface 13 of said head portion 11 has an inclination of above 9° with respect to said surface 5 of said base plate 3, then the projections 7 may invade the skin, and the projections 7 may be broken or bent without springing back to their original shape after swaying.

Said flat top surface 13 of said head portion 11 covers above 85% ofthe surface area of said head portion 11 excluding said side 15.

If said flat top surface 13 of said head portion 11 covers below 85% of the surface area of said head portion 11 excluding said side 15, then the projections 7 may invade the skin, and the projections 7 may be broken or bent without springing back to their original shape after swaying.

If said flat top surface 13 of said head portion 11 covers above 85% of the surface area of said head portion 11 excluding said side 15, then a curved surface 23 may exist between said flat top surface 13 of said head portion 11 and said side 15 of said head portion 11. See FIG. 5.

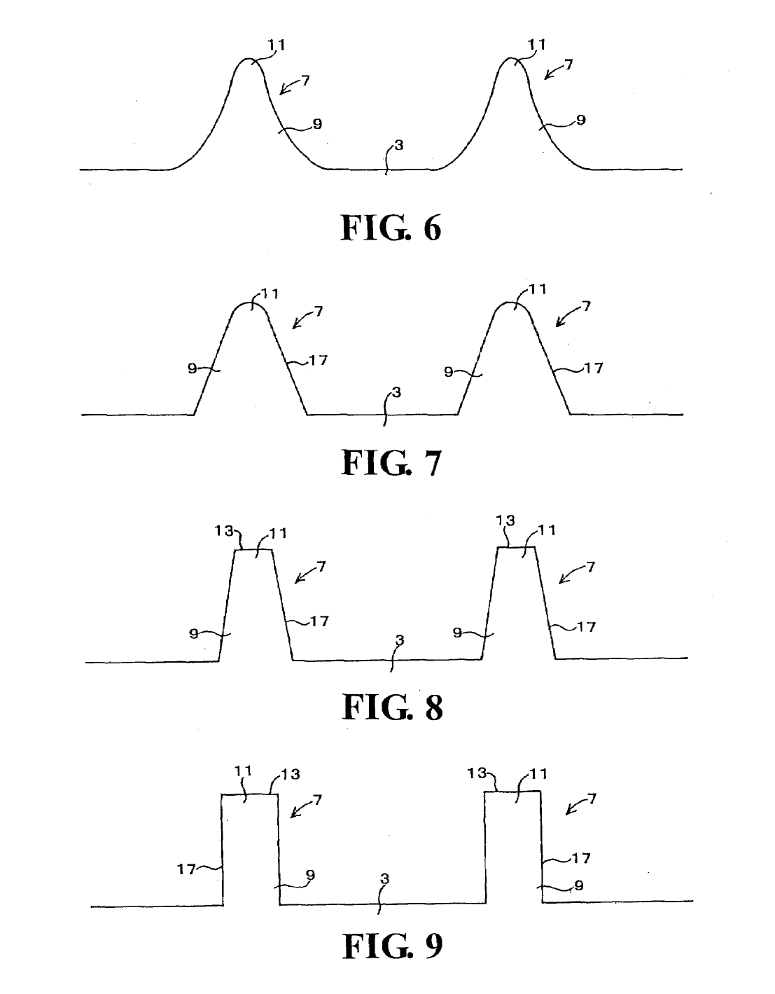

Projections 7 shown in FIG. 6 may invade the skin, and the projections 7 may be broken or bent without springing back to their original shape after swaying because the head portion 11 does not have a flat top surface 13. The projections 7 shown in FIG. 6 do not sway well because the head portion 11 does not have a side 15 perpendicular to said surface 5 of said base plate 3.

Projections 7 shown in FIG. 7 may invade the skin and do not sway because the head portion 11 does not have a flat top surface 13, the head portion 11 does not have a side 15 perpendicular to said surface 5 of said base plate 3, and said base portion 9 does not have an inwardly depressed external curved line 17 in a central vertical section.

Projections 7 shown in FIG. 8 maybe broken or bent without swaying because the projections 7 have an overall shape of a truncated cone.

Projections 7 shown in FIG. 9 maybe broken or bent without swaying because the projections 7 have an overall shape of a cylinder.

Said base plate 3 and projections 7 are made of a material having a surface hardness of 80 to 120 by Rockwell hardness test or durometer hardness test.

Materials usable for making the base plate 3 and projections 7 include polyamide, polymethylpentene, silicone, elastomer, polyacetal,thermoplastic polyurethane elastomer, vinyl chloride, polypropylene and polyethylene. Particularly, polyamide, polymethylpentene, silicone and elastomer are preferably used.

If the surface hardness of the material by the Rockwell hardness test or durometer hardness test exceeds 120, then the projections 7 may be broken. Even though the head portion 11 has the flat top surface 13, the projections 7 may invade the skin when the acupuncture needle is in contact with or pressed against the skin 21.

If the surface hardness of the material by Rockwell hardness test or durometer hardness test is below 80, then the projections 7 may be bent and lose their original form when the acupuncture needle is in contact with or pressed against the skin 21.

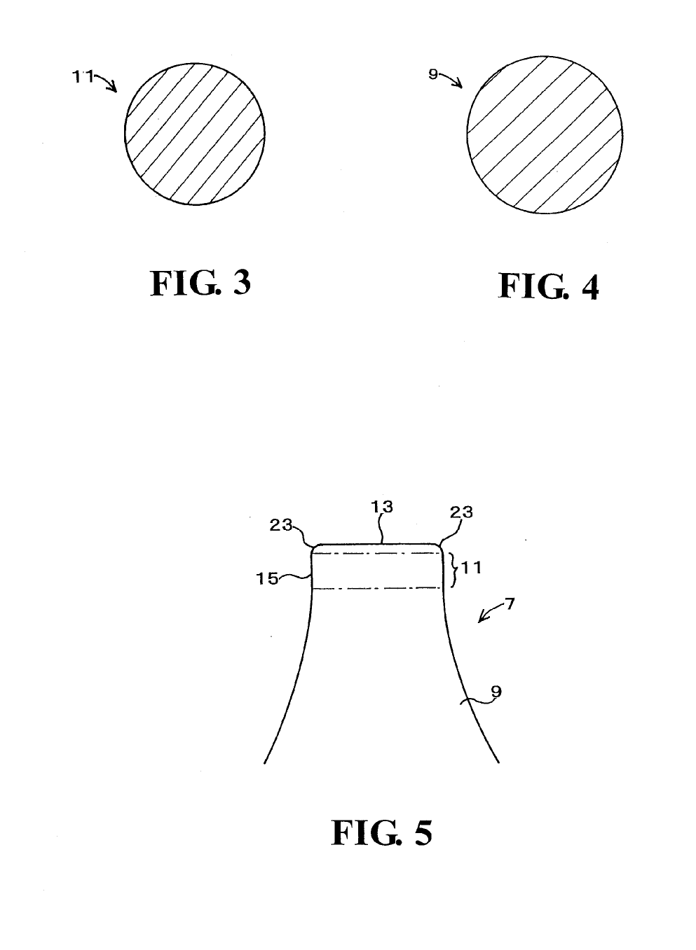

The head portion 11 of each projection 7 is formed substantially cylindrical and is circular in a plan view. See FIG. 3.

If the head portion 11 of the projection 7 has a triangular, square, hexagonal or other angular shape or an oval shape in a plan view, the head portion 11 may invade the skin 21 because the head portion 11 does not exert force uniformly on the contact surface when the head portion 11 is in contact with the skin 21.

The head portion 11 has a diameter of 10 to 70 μm.

If the head portion 11 has a diameter of below 10 μm, then the head portion 11 may invade the skin 21 and the projection 7 may be broken or bent.

If the head portion 11 has a diameter of above 70 μm, the projections 7 may not sway when the acupuncture needle 1 is in contact with or pressed against the skin 21 because the area of the top surface 13 of the head portion 11 is too large.

The distance between said surface 5 of the base plate 3 and the top surface 13 of the head portion 11 is 50 to 500 μm.

If the distance between said surface 5 of the base plate 3 and the top surface 13 of the head portion 11 is below 50μm, and the diameter of the head portion 11 is 10 to 70 μm, then the projections 7 may not sway when the acupuncture needle 1 is in contact with or press against the skin 21. In this case, the projections 7 may invade the skin 21.

If the distance between said surface 5 of the base plate 3 and the top surface 13 of the head portion 11 is above 500 μm, and the diameter of the head portion 11 is 10 to 70 μm, then the projections 7 may be broken or bent and the projections 7 may not sway when the acupuncture needle 1 is in contact with or pressed against the skin 21. In this case, the projections 7 may invade the skin 21.

The length of said side 15 of the head portion 11 perpendicular to said surface 5 of the base plate 3 is 5 to 50 μm and below 10% of said distance (50 to 500 μm) between said surface 5 of the base plate 3 and the top surface 13 of the head portion 11.

Only when the length of said side 15 of the head portion 11 perpendicular to said surface 5 of the base plate 3 is within said range, the projections 7 sway.

The acupuncture needle 1 of the present invention may be attached to a supporting member 33 having a handle 31 as shown in FIGS. 11 and 12. In this case, the handle 31 is held with the hand and the acupuncture needle 1 attached to the supporting member 33 is brought into contact with or pressed against the skin 21.

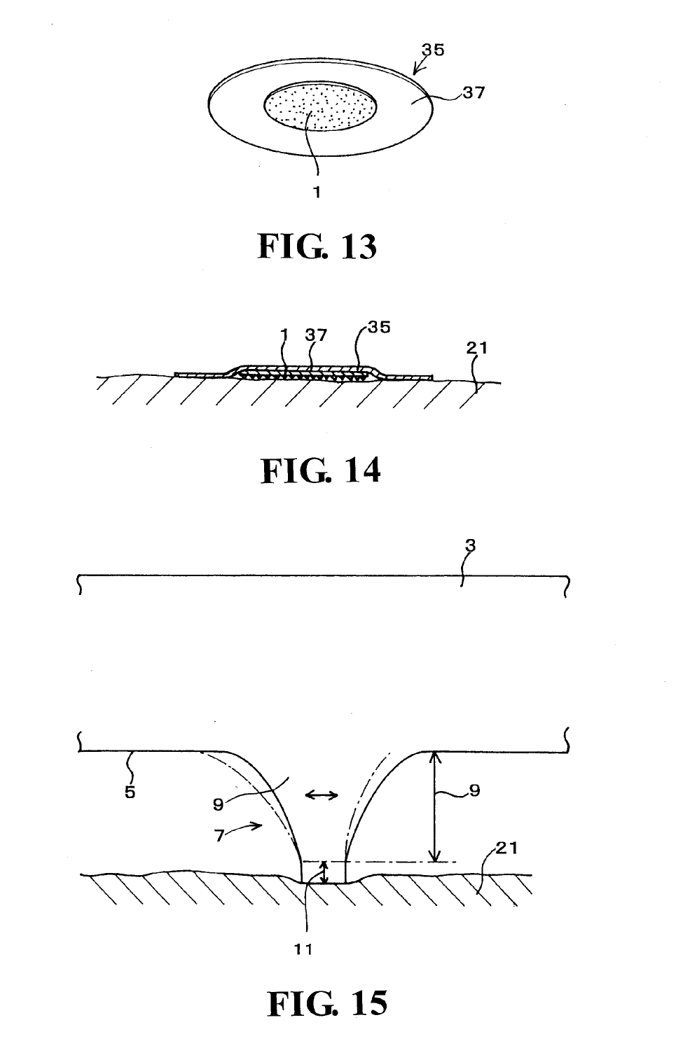

Alternatively, the acupuncture needle 1 of the present invention may be attached to an adhesive surface 37 of an adhesive sheet 35 such as an adhesive plaster as shown in FIGS. 13 and 14. In this case, the acupuncture needle 1 is brought into contact with or pressed against the skin 21 by sticking the adhesive sheet 35 on the skin 21.

As many apparently widely different embodiments of the present invention may be made without departing from the spirit and scope thereof, it is to be understood that the invention is not limited to the specific embodiments thereof except as defined in the appended claims.

1. An acupuncture needle comprising:
a base plate having a surface; and
a plurality of projections formed on said surface of said base plate integrally therewith, each of said projections having elasticity and comprising a base portion and a substantially cylindrical head portion formed on said base portion, said head portion having a flat top surface substantially parallel to said surface of said base plate and a side perpendicular to said surface of said base plate, said base portion having an inwardly depressed external curved line in a central vertical section and a diameter in a transverse cross section gradually becoming larger toward said surface of said base plate.

2. An acupuncture needle as claimed in claim 1, wherein said flat top surface of said head portion has an inclination of below 9° with respect to said surface of said base plate.

3. An acupuncture needle as claimed in claim, wherein said flat top surface of said head portion covers above 85% of surface area of said head portion excluding said side.

4. An acupuncture needle as claimed in claim 1, wherein said base plate and said projections are comprised of a material having a surface hardness of 80 to 120 by Rockwell hardness test or durometer hardness test.

5. An acupuncture needle as claimed in claim 1, wherein said head portion has a diameter of 10 to 70 μm.

6. An acupuncture needle as claimed in claim 1, wherein distance between said surface of said base plate and said top surface of said head portion is 50 to 500 μm.

7. An acupuncture needle as claimed in claim 6, wherein length of said side of said head portion perpendicular to said surface of said base plate is 5 to 50 μm and below 10% of said distance between said surface of said base plate and said top surface of said head portion.
